(19) United States
(12) Plant Patent Application Publication
(10) Pub. No.: US 2025/0081866 P1
(43) Pub. Date: Mar. 6, 2025

(54) APPLE PLANT NAMED 'BENITSURUGI'

(50) ;

(71) Applicant: NATIONAL AGRICULTURE AND FOOD RESEARCH ORGANIZATION, Tsukuba (JP)

(72) Inventors: Yutaka Sawamura, Morioka (JP); Shigeki Moriya, Bunkyo-ku (JP); Kazuyuki Abe, Funagata-machi (JP); Kazuma Okada, Tsukuba (JP); Taku Shimizu, Morioka (JP); Hiroshi Iwanami, Morioka (JP); Nobuhiro Kotoda, Saga (JP); Takashi Haji, Tsukuba (JP); Sae Takahashi, Utsunomiya (JP); Yuki Moriya, Morioka (JP); Hirohisa Nesumi, Tsukuba (JP); Ayato Hori, Morioka (JP); Takashi Baba, Morioka (JP)

(73) Assignee: NATIONAL AGRICULTURE AND FOOD RESEARCH ORGANIZATION, Tsukuba (JP)

(21) Appl. No.: 18/789,972

(22) Filed: Jul. 31, 2024

(30) Foreign Application Priority Data
Aug. 31, 2023 (JP) PVP37015

(51) Int. Cl. A01H 6/74

(52) U.S. Cl. CPC A01H 6/7418
USPC PLT/161

(57) ABSTRACT

A new variety of apple tree ‘BENITSURUGI’ that has columnar tree type, bearing on spurs only, thick to very thick shoot thickness, short internode length, long leaf blade length, long petiole length, short to medium fruit stalk length, medium size and short to medium height fruit with fully open locules.

Common name: Apple.

Botanical classification: Malus domestica (Suclow) Borkh.

Variety denomination: ‘BENITSURUGI’.

### CROSS-REFERENCE TO RELATED APPLICATION

This application claims priority of Japanese plant variety rights application No. 37015filed Aug. 31, 2023, the entire content of which is incorporated by reference herein.

### BACKGROUND OF THE NEW VARIETY

The present invention relates to a new and distinct variety of apple tree, Malus domestica (Suclow) Borkh, which has been given the variety denomination ‘BENITSURUGI’.

This new apple tree has a columnar tree type, bearing on spurs only, thick to very thick shoot thickness, short internode length, long leaf blade length, long petiole length, short to medium fruit stalk length, medium size and short to medium height fruit with fully open locules, that distinguish it, notably from its male parent ‘5-12786’ (medium extent of anthocyanin overcolor of young fruit, late harvest time) and female parent ‘SANSA’ (ramified tree type, medium shoot thickness, medium internode length, early harvest time), as well as from reference varieties ‘SENSHU’ (ramified tree type, bearing on spurs and long shoots, thin to medium shoot thickness, medium internode length, medium leaf blade length, short to medium petiole length, fruit with moderately open locules) and ‘SHINANO SWEET’ (ramified tree type, bearing on spurs and long shoots, thin to medium shoot thickness, medium internode length, short to medium leaf blade length, short to medium petiole length, medium to long fruit stalk length, large size and tall height fruit).

### ORIGIN OF THE VARIETY

The parent varieties are ‘5-12786’ (male parent) (not patented) and ‘SANSA’ (female parent) (not patented), respectively.

The variety was developed and propagated at the Morioka Research Station, Institute of Fruit Tree and Tea Science, National Agriculture and Food Research Organization (NARO), in Morioka, Iwate, Japan.

The variety was reproduced asexually by vegetative reproduction by grafting one-year-old cuttings on rootstocks.

In 2005, crossing the parent varieties and collecting seeds.

In 2006, columnar type's seedlings are selected by SCAR682 DNA marker from growing seedlings.

In 2007, they were grafted on JM1 root stock.

In 2008, they were transplanted at breeding field. And selected individual plant.

In 2015, the plant named Morioka 74 as a promising breeding line.

In 2016, growing test for local adaptability were started under confidentiality conditions at 16 public testing stations in Japan.

On Aug. 31, 2023, an application for plant variety rights No. 37015 was filed for the present variety in the Japanese Plant Variety Protection Office of the Ministry of Agriculture, Forestry and Fisheries (MAFF), Japan.

Cultivation of the variety does not require special conditions or treatments.

The variety is currently mainly intended for fruit production (table use).

### SUMMARY OF THE VARIETY

The present variety is distinguished by columnar tree type, bearing on spurs only, thick to very thick shoot thickness, short internode length, long leaf blade length, long petiole length, short to medium fruit stalk length, medium size and short to medium height fruit with fully open locules, that distinguish it, notably from its male parent ‘5-12786’ (medium extent of anthocyanin overcolor of young fruit, late harvest time) and female parent ‘SANSA’ (ramified tree type, medium shoot thickness, medium internode length, early harvest time), as well as from reference varieties ‘SENSHU’ (ramified tree type, bearing on spurs and long shoots, thin to medium shoot thickness, medium internode length, medium leaf blade length, short to medium petiole length, fruit with moderately open locules) and ‘SHINANO SWEET’ (ramified tree type, bearing on spurs and long shoots, thin to medium shoot thickness, medium internode length, short to medium leaf blade length, short to medium petiole length, medium to long fruit stalk length, large size and tall height fruit).

A comparison with the male parent ‘5-12786’ is as follows (evaluation based on averages):

TABLE 1

| | | Male parent | Present variety |
|---|---|---|---|
| | | ‘5-12786’ | ‘BENITSURUGI’ |
| No. | Characteristics | States | States |
| 23 | Young fruit: extent | medium | absent or very |
| | of anthocyanin | | small to small |
| | overcolor | | |
| 63 | Time of harvest | late | medium |

A comparison with the female parent ‘SANSA’ is as follows (evaluation based on averages):

TABLE 2

| | | Female parent | Present variety |
|---|---|---|---|
| | | ‘SANSA’ | ‘BENITSURUGI’ |
| No. | Characteristics | States | States |
| 2 | Tree: type | ramified | columnar |
| 5 | One-year-old shoot: | medium | thick to |
| | thickness | | very thick |
| 6 | One-year-old shoot: | medium | short |
| | length of internode | | |
| 63 | Time of harvest | early | medium |

A comparison with the reference varieties ‘SENSHU’ and ‘SHINANO SWEET’ is as follows (evaluation based on averages):

TABLE 3

| | | | Reference | |
|---|---|---|---|---|
| | | Reference | variety | Present |
| | | variety | ‘SHINANO | variety |
| | | ‘SENSHU’ | SWEET’ | ‘BENITSURUGI’ |
| No. | Characteristics | States | States | States |
| 2 | Tree: type | ramified | ramified | columnar |
| 4 | Tree: type | on spurs and | on spurs and | on spurs only |
| | of bearing | long shoots | long shoots | |
| 5 | One-year-old shoot: | thin to | thin to | thick to |
| | thickness | medium | medium | very thick |
| 6 | One-year-old shoot: | medium | medium | short |
| | length of internode | | | |
| 11 | Leaf blade: length | medium | short to | long |
| | | | medium | |
| 12 | Leaf blade: width | narrow to | narrow to | medium to |
| | | medium | medium | broad |
| 17 | Petiole: length | short to | short to | long |
| | | medium | medium | |
| 18 | Petiole: extent of | | medium | small |
| | anthocyanin | | | |
| | coloration | | | |
| | from base | | | |
| 19 | Flower: | | light pink | dark pink |
| | predominant | | | |
| | color at | | | |
| | balloon stage | | | |
| 23 | Young fruit: | large | | absent or very |
| | extent of | | | small to small |
| | anthocyanin | | | |
| | overcolor | | | |
| 24 | Fruit: size | | large | medium |
| 25 | Fruit: height | | tall | short to medium |
| 49 | Fruit: length | medium | medium | short to medium |
| | of stalk | | to long | |
| 61 | Fruit: aperture | moderately | | fully open |
| | of locules | open | | |
| | (in transverse | | | |
| | section) | | | |
| 63 | Time of harvest | | medium | medium |
| | | | to late | |

### BRIEF DESCRIPTION OF THE DRAWINGS

In the accompanying drawings, which are as nearly true as is reasonable possible to make in a color illustration of this type:

FIG. 1 is a color photograph showing a general view of the apple tree named ‘BENITSURUGI’ at harvest time.

FIG. 2 is a color photograph showing a flower of ‘BENITSURUGI’.

FIG. 3 is a color photograph showing respective parts of flower of ‘BENITSURUGI’.

FIG. 4 is a color photograph showing fruits of ‘BENITSURUGI’.

FIG. 5 is a color photograph showing cross sections of fruits of ‘BENITSURUGI’.

FIG. 6A is a color photograph showing the tree type of reference variety ‘SHINANO SWEET’.

FIG. 6B is a color photograph showing the tree type of reference variety ‘SENSHU’.

FIG. 6C is a color photograph showing the tree type of the present variety ‘BENITSURUGI’.

FIG. 7 is a color photograph showing one year old shoot of ‘BENITSURUGI’ (top), in comparison with ‘SENSHU’ (middle) and ‘SHINANO SWEET’ (bottom).

FIG. 8 is a color photograph showing leaves of ‘BENITSURUGI’ (center), in comparison with ‘SHINANO SWEET’ (left) and ‘SENSHU’ (right).

FIG. 9A is a color photograph showing the petiole: extent of anthocyanin coloration from base of ‘BENITSURUGI’ (bottom), in comparison with ‘SHINANO SWEET’ (top).

FIG. 9B is a color photograph showing a more detailed view of the petiole: extent of anthocyanin coloration from base of ‘BENITSURUGI’ (bottom), in comparison with ‘SHINANO SWEET’ (top).

FIG. 10 is a color photograph showing the flower bud of ‘BENITSURUGI’ (left), in comparison with ‘SHINANO SWEET’ (right).

FIG. 11 is a color photograph showing the young fruit of ‘BENITSURUGI’ (left), in comparison with ‘SENSHU’ (right).

FIG. 12 is a color photograph showing the fruit of ‘BENITSURUGI’ (center), in comparison with ‘SHINANO SWEET’ (top) and ‘SENSHU’ (bottom).

FIG. 13 is a color photograph showing another view of the fruit of ‘BENITSURUGI’ (center), in comparison with ‘SENSHU’ (left) and ‘SHINANO SWEET’ (right).

FIG. 14 is a color photograph showing the underside of fruit of ‘BENITSURUGI’ (middle), in comparison with ‘SHINANO SWEET’ (top) and ‘SENSHU’ (bottom).

FIG. 15 is a color photograph showing the fruit stalk of ‘BENITSURUGI’ (center), in comparison with ‘SENSHU’ (left) and ‘SHINANO SWEET’ (right).

FIG. 16 is a color photograph showing the fruit shape of core of ‘BENITSURUGI’ (middle), in comparison with ‘SHINANO SWEET’ (top) and ‘SENSHU’ (bottom).

FIG. 17 is a color photograph showing the aperture of locules in transverse section of the fruit of ‘BENITSURUGI’ (right), in comparison with ‘SENSHU’ (left).

FIG. 18 is a color photograph showing a general view of the fruit of ‘BENITSURUGI’.

Due to chemical and/or digital development, processing and printing, the plants or portions of plants depicted in the photographs may or may not be precisely accurate, when compared to the actual botanical specimens.

### DETAILED DESCRIPTION OF THE INVENTION

The botanical description and the photographs are of the ‘BENITSURUGI’ plants evaluated from 2020 to 2022 (including for the comparison with the reference plants ‘SENSHU’ and ‘SHINANO SWEET’. Values as provided are averages.

The plants shown on the photographs were grown at Morioka. Iwate, Japan. The dates each photograph was taken are as follows:

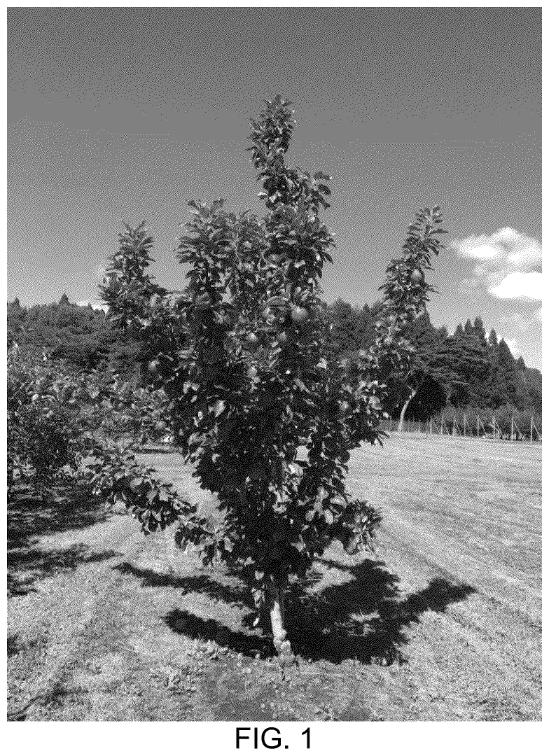

### FIGS. 1, 18: Oct. 2, 2020,

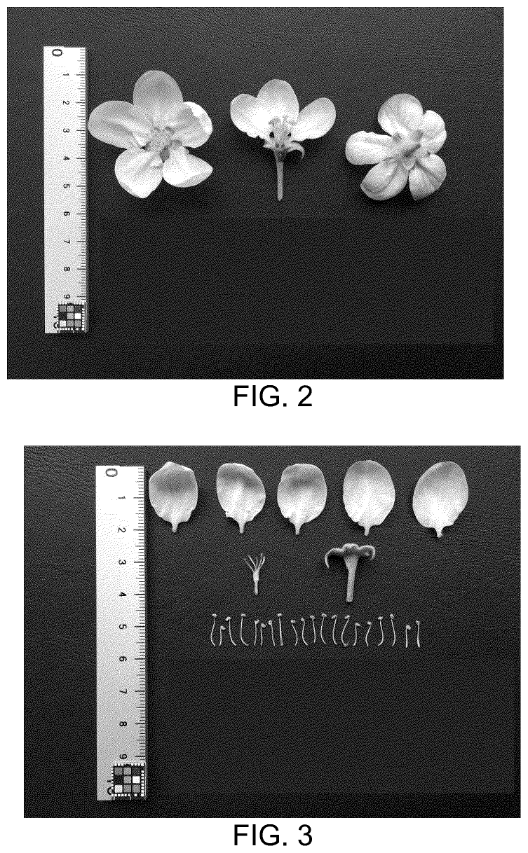

### FIGS. 2, 3: May 11, 2020

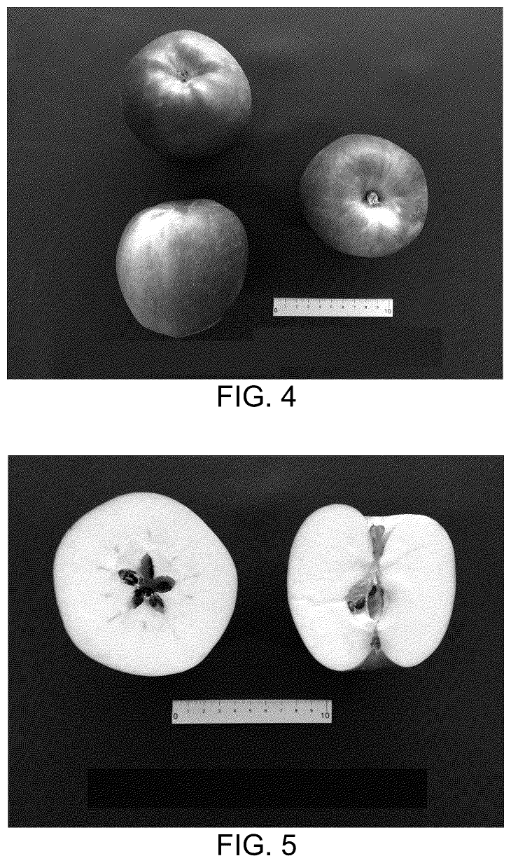

### FIGS. 4, 5: Oct. 9, 2020

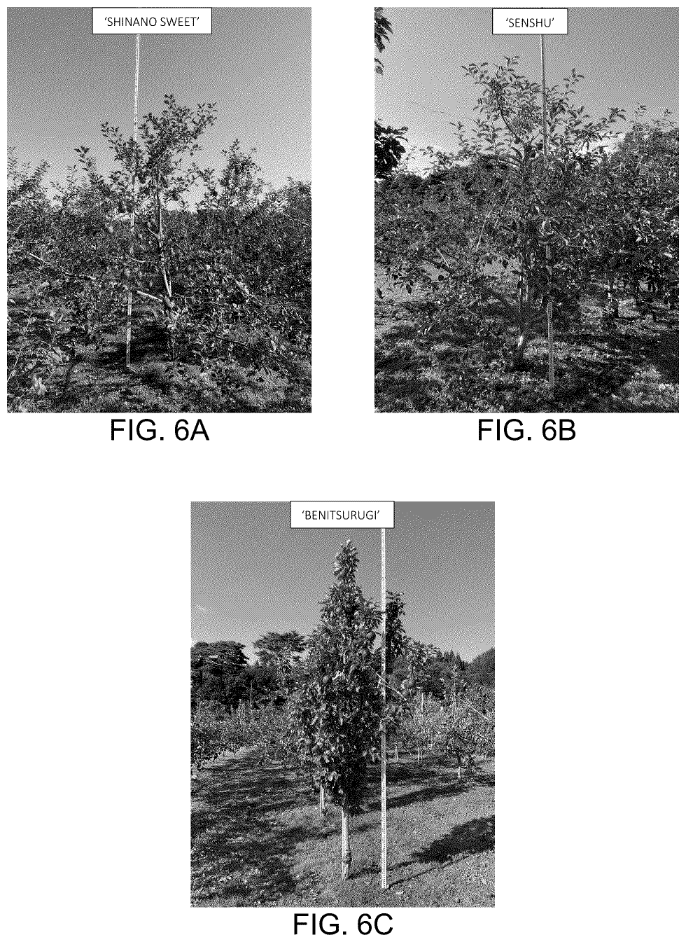

### FIGS. 6A, 6B, 6C: Sep. 29, 2022

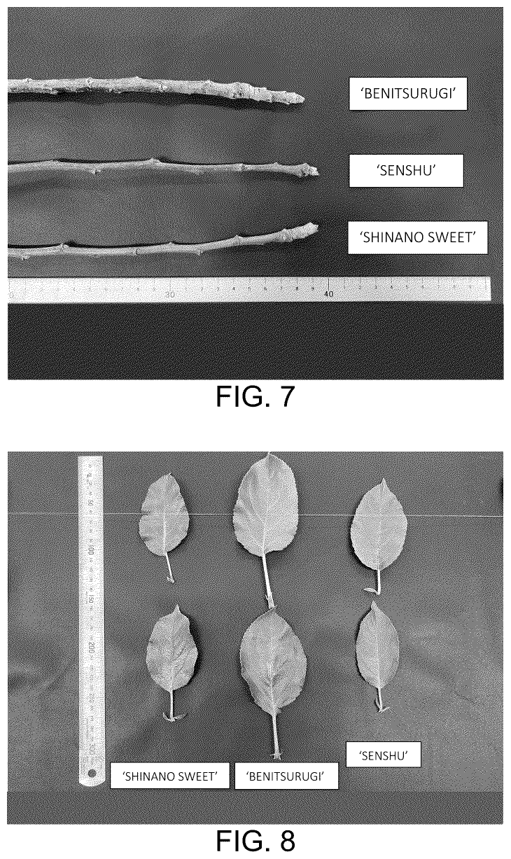

### FIG. 7: Dec. 23, 2022

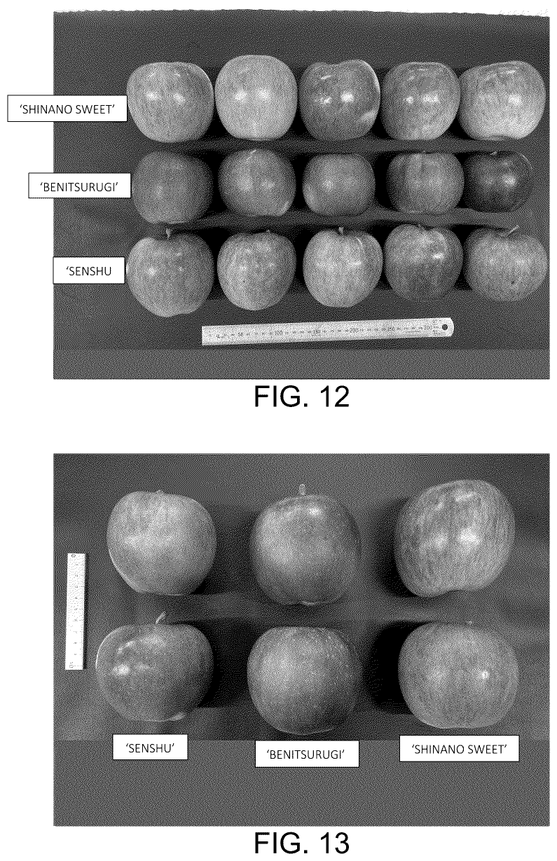

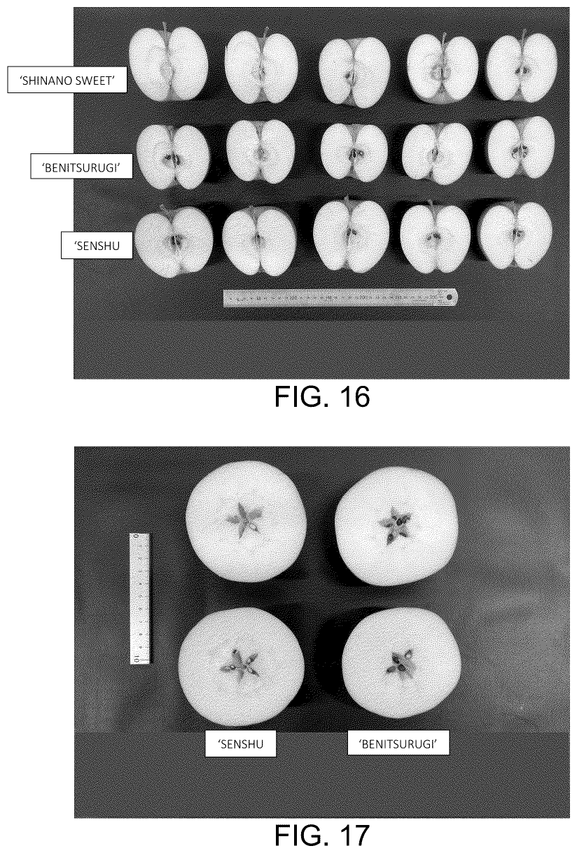

### FIGS. 8, 12, 16: Dec. 15, 2022

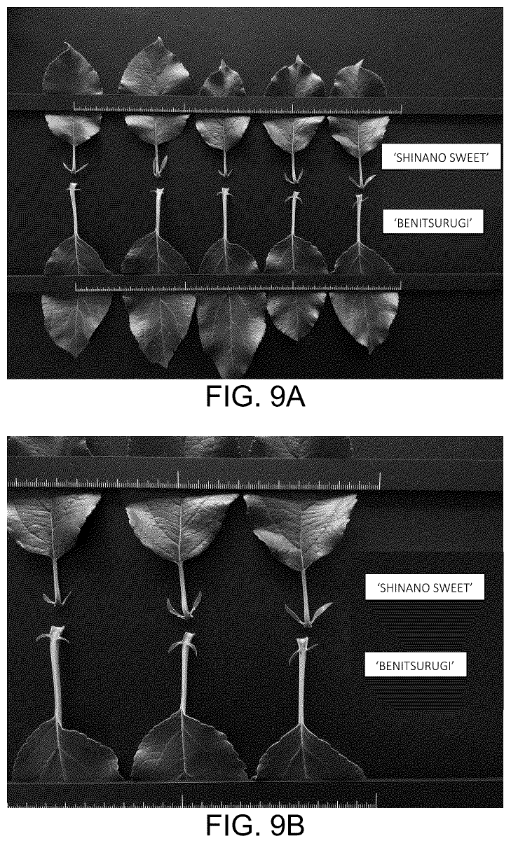

### FIG. 9A, 9B: Aug. 24, 2020

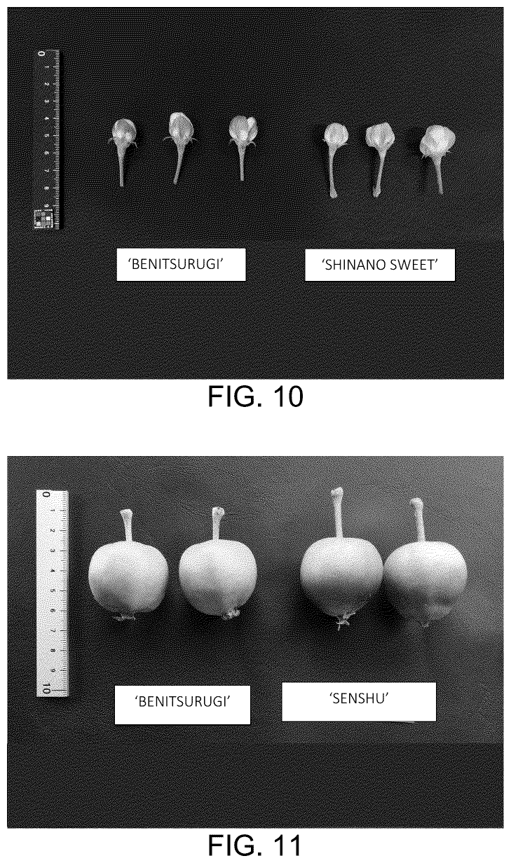

### FIG. 10: May 10, 2021

### FIG. 11: Jun. 21, 2021

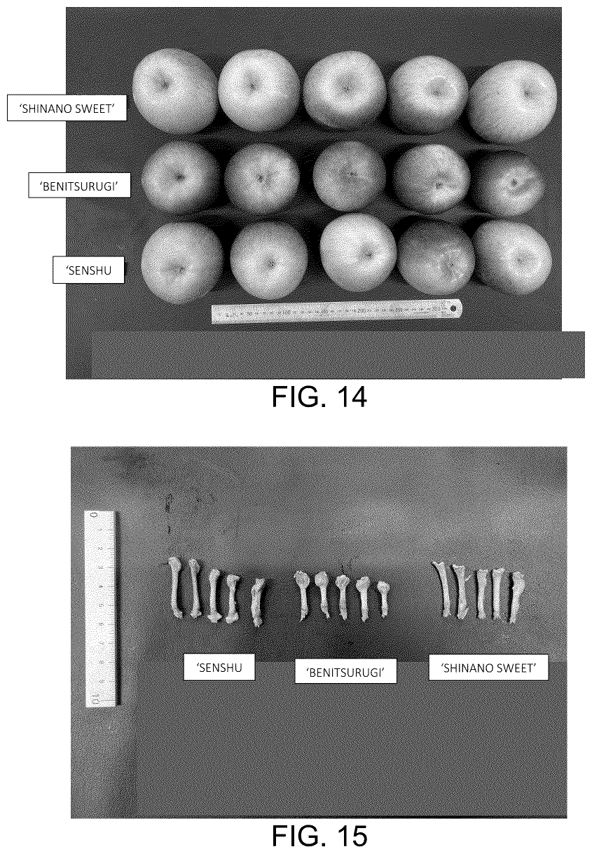

### FIGS. 13, 14, 15, 17: Dec. 1, 2021

Colors are given according to the R.H.S. (Royal Horticultural Society) Color Chart.

### BOTANICAL DESCRIPTION

TABLE 4

| No. | Characteristics | States | Values | |
|---|---|---|---|---|
| 1 | Tree: vigor | medium | | |
| 2 | Tree: type | columnar | | |
| 3 | Only varieties with ramified tree type: | not applicable | | |
| | Tree: habit | | | |
| 4 | Tree: type of bearing | on spurs only | | |
| 5 | One-year-old shoot: thickness | thick to very thick | 8.2 | mm |
| 6 | One-year-old shoot: length of internode | short | 1.2 | cm |
| 7 | One-year-old shoot: color on sunny side | medium brown | 200A | |
| | | | (dark brown) | |
| 8 | One-year-old shoot: pubescence (on | strong | | |
| | distal half of shoot) | | | |
| 9 | One-year-old shoot: number of lenticels | many | | |
| 10 | Leaf blade: attitude in relation to shoot | outwards | | |
| 11 | Leaf blade: length | long | 11.5 | cm |
| 12 | Leaf blade: width | medium to broad | 6.6 | cm |

| 13 | Leaf blade: ratio length/width | medium to large | 1.7 | |
|---|---|---|---|---|
| 14 | Leaf blade: intensity of green color | medium | 139A | |
| | | | (dark green) | |
| 15 | Leaf blade: incisions of margin | serrate type 2 | | |
| | (upper half) | | | |
| 16 | Leaf blade: pubescence on lower side | medium | | |
| 17 | Petiole: length | long | 4.6 | cm |

| 18 | Petiole: extent of anthocyanin | small | 22.50% | |
|---|---|---|---|---|
| | coloration from base | | | |
| 19 | Flower: predominant color at balloon | dark pink | N74C | |
| | stage | | (blue pink) | |
| 20 | Flower: diameter with petals pressed | small to medium | 4.8 | cm |
| | into horizontal position | | | |
| 21 | Flower: arrangement of petals | overlapping | | |
| 22 | Flower: position of stigmas relative to | same level | | |
| | anthers | | | |
| 23 | Young fruit: extent of anthocyanin | absent or very | 10.70% | |
| | overcolor | small to small | | |
| 24 | Fruit: size | medium | 311.5 | g |
| 25 | Fruit: height | short to medium | 8 | cm |
| 26 | Fruit: diameter | medium | 9.1 | cm |
| 27 | Fruit: ratio height/diameter | medium | 0.88 | |
| 28 | Fruit: general shape | globose | | |
| 29 | Fruit: ribbing | absent or weak | | |
| 30 | Fruit: crowning at calyx end | absent or weak | | |
| 31 | Fruit: size of eye | very small to small | | |
| 32 | Fruit: length of sepal | short to medium | | |
| 33 | Fruit: bloom of skin | absent or weak | | |
| 34 | Fruit: greasiness of skin | absent or weak | | |
| 35 | Fruit: surface texture of skin | smooth | | |
| 36 | Fruit: ground color | yellow green | 150 | C. |
| | | | (yellow green) | |
| 37 | Fruit: relative area of over color | large | 87.7% | |
| 38 | Fruit: hue of over color-with bloom | red | 187B | |
| | removed | | (dark purple red) | |
| 39 | Fruit: intensity of over color | dark | | |
| 40 | Fruit: pattern of over color | solid flush with | | |
| | | weakly defined | | |
| | | stripes | | |
| 41 | Fruit: width of stripes | narrow to medium | | |
| 42 | Fruit: intensity of stripes | weak to medium | | |
| 43 | Fruit: area of russet around stalk | absent or small | | |
| | attachment | | | |
| 44 | Fruit: area of russet on cheeks | absent or small | | |
| 45 | Fruit: area of russet around eye basin | absent or small | | |
| 46 | Fruit: number of lenticels | medium | | |
| 47 | Fruit: size of lenticels | small to medium | | |
| 48 | Fruit: scarfskin | very small to | | |
| | | small | | |
| 49 | Fruit: length of stalk | short to medium | 1.8 | cm |
| 50 | Fruit: thickness of stalk | thin to medium | 3.1 | mm |
| 51 | Fruit: depth of stalk cavity | shallow medium | 2.2 | cm |
| 52 | Fruit: width of stalk cavity | narrow medium | 4 | cm |
| 53 | Fruit: depth of eye basin | medium | 1.2 | cm |
| 54 | Fruit: width of eye basin | medium | 3.4 | cm |
| 55 | Fruit: firmness of flesh | medium | 15 | lbs |

| 56 | Fruit: color of flesh | cream | 8D | |
|---|---|---|---|---|
| | | | (light yellow) | |
| 57 | Fruit: sweetness of flesh | medium | 13.5% | |
| 58 | Fruit: acidity of flesh | low | 0.36 | g/ |
| | | | 100 | ml |

| | | | |
|---|---|---|---|
| 59 | Fruit: water-core of flesh | absent or very slight | |
| 60 | Fruit: shape of core | flat round | |
| 61 | Fruit: aperture of locules (in transverse | fully open | |
| | section) | | |
| 62 | Time of beginning of flowering | early to medium | May 9 |
| 63 | Time of harvest | medium | October 2 |
| 64 | Resistance to: Alternalia blotch | strong | |

Other features of the plant are as follows:
   Disease resistance.—Resistance to Alternaria blotch (Alternaria altemata) was observed. The new variety has the same susceptibility to Blotch (Diplocarpon mali), Powdery mildew (Podosphaera leucotricha) and Scab (Venturia inaequalis) as existing cultivars.
      I/we claim:

1. A new and distinct variety of apple tree named ‘BENITSURUGI’, substantially as described and illustrated herein.
